(19) United States
(12) Patent Application Publication
(10) Pub. No.: US 2020/0138357 A1
(43) Pub. Date: May 7, 2020

(54) ARTIFICIAL IMMUNITY BRAIN COMPUTER INTERFACE SYSTEM

(71) Applicant: University of Louisiana at Lafayette, Lafayette, LA (US)

(72) Inventors: Magdy Bayoumi, Lafayette, LA (US); Zaghloul Saad Elsayed, Lafayette, LA (US)

(21) Appl. No.: 16/671,239

(22) Filed: Nov. 1, 2019

Related U.S. Application Data
(60) Provisional application No. 62/753,996, filed on Nov. 1, 2018.

(51) Int. Cl. A61B 5/18; G06F 3/01; A61B 5/00; A61B 5/0476; A61B 5/0402; A61B 5/0496; A61B 5/0488; A61B 5/11; A61B 5/145

(52) U.S. Cl. CPC A61B 5/18; G06F 3/015; A61B 5/6803; A61B 5/0476; G05D 1/0055; A61B 5/0496; A61B 5/0488; A61B 5/11; A61B 5/14546; A61B 5/0402

(57) ABSTRACT

Driving while feeling drowsy can cause fatal accidents. Current drowsiness detection devices are slow and inefficient. Mental drowsiness detection can predict a fatigue state early enough to engage the autopilot mechanism. Disclosed is a wearable EEG BCI adaptive VLSI architecture that can detect sleep faster.

### Cross Reference to a Related Application

This application claims priority to the Provisional U.S. patent application Ser. No. 62/753,996 entitled “Artificial Immunity Brain Computer Interface System” filed Nov. 1, 2018.

### STATEMENT REGARDING FEDERALLY SPONSORED RESEARCH OR DEVELOPMENT

Not applicable.

### REFERENCE TO A “SEQUENCE LISTING”, A TABLE, OR COMPUTER PROGRAM

Not applicable.

### DESCRIPTION OF THE DRAWINGS

The drawings constitute a part of this specification and include exemplary examples of the ARTIFICIAL IMMUNITY BRAIN COMPUTER INTERFACE SYSTEM, which may take the form of multiple embodiments. It is to be understood that, in some instances, various aspects of the invention may be shown exaggerated or enlarged to facilitate an understanding of the invention. Therefore, drawings may not be to scale.

FIG. 1 provides the ARTIFICIAL IMMUNITY BRAIN COMPUTER INTERFACE SYSTEM design and the architecture block diagram.

FIG. 2 provides the ARTIFICIAL IMMUNITY BRAIN COMPUTER INTERFACE SYSTEM component of the alpha waves attenuation counter block diagram.

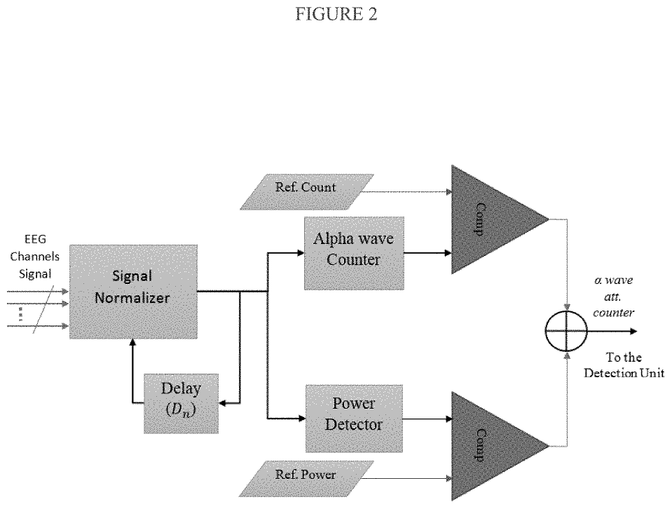

FIG. 3 provides the ARTIFICIAL IMMUNITY BRAIN COMPUTER INTERFACE SYSTEM detection unit logical block diagram.

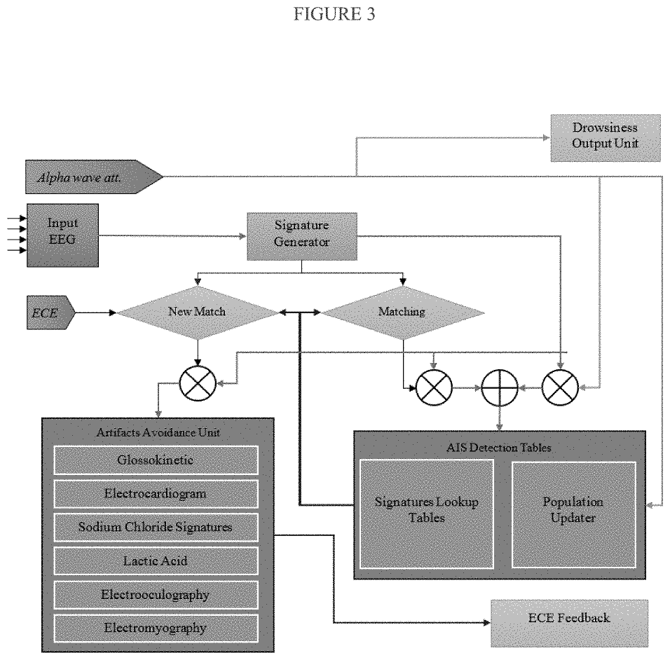

FIG. 4 provides the ARTIFICIAL IMMUNITY BRAIN COMPUTER INTERFACE SYSTEM the headset design and concept diagram.

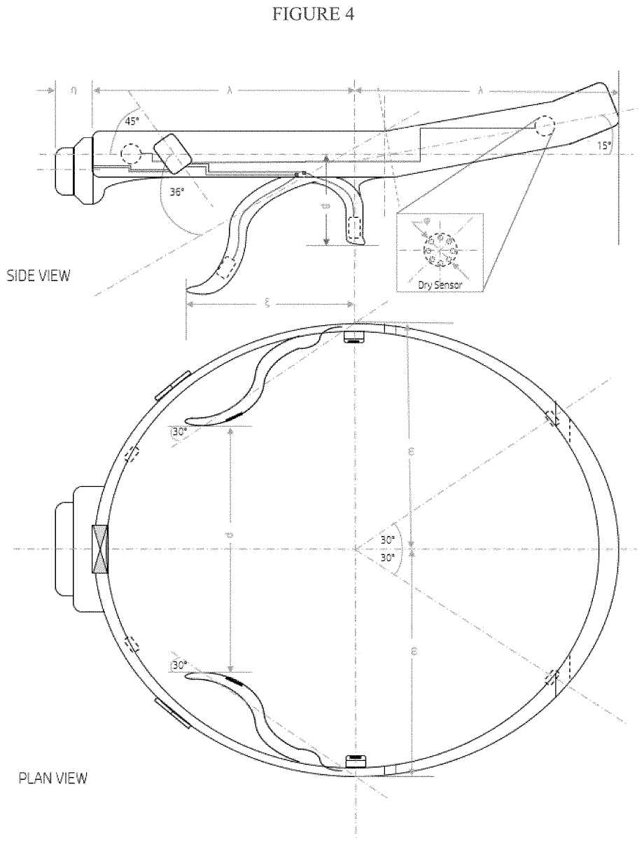

### FIELD OF THE INVENTION

The invention generally relates to the field of systems for pattern detection and artifacts avoidance in human movement, which earlier predicts sleep and the beginning of drowsiness; namely, the use of these patterns to detect and prevent injury based upon detecting drowsiness.

### BACKGROUND OF THE INVENTION

Autonomous cars are a dream that will be available in the near future. At present, its concepts have not been perfected. However, its current technology can be very helpful as a driver assistance aid, particularly in life threatening situations. According to the National Highway Traffic Safety Administration, in the United States alone, eleven million drivers admit they have had an accident because they fell asleep. This results in an estimated 1,550 deaths, 71,000 injuries, and $12.5 billion in losses.

The currently available sleep detection devices are based upon eye movements, head position, and image processing algorithms, which is usually computationally (See B, M Kumari, P. Kumar, “A survey on drowsy driver detection system,” IEEE Int. Conf. on Big Data Analytics and Com. (ICBDAC) 2017); however, the eye closed event detection is typically too late to avoid fatal consequences, particularly on highways.

Metal drowsiness occurs earlier than or independent of eye fatigue, which can be detected via Electroencephalography (EEG). Most of EEG processing requires large hardware and suffers from low accuracy (<60%) due to the signal noises and the BCI artifacts that make it a significant task (See N. Elsayed, M. Bayoumi, “Brain computer interface: EEG signal preprocessing issues and solutions,” IJCA, vol. 169, no. 3, July 2017).

The disclosed invention describes a wearable, easy-to-use, noise-proof BCI architecture. Using novel parameterized adaptive artificial immunity system (AIS) for pattern detection and artifacts avoidance, which earlier predicts sleep and the beginning of drowsiness (˜1.49 sec) via a dual real-time brain-wave scanning VLSI technique, with low false positive rate (FRP), 96.54% accuracy, which connects to the autonomous car auto pilot system that can save a human life.

Recent research and commercial products were introduced for drowsy driver detection, using head position, face detection, eye tracing, infrared scanning, even skin resistance, but most of them suffer from low accuracy and late alerts with a high false positive rate (FPR). The disclosed invention focuses on EEG based methods. While EEG signals are very noisy, the attenuation of the EEG Alpha rhythm (8-12 Hz, 20-200 uV) is the most valid marker of the sleep onset period and concentration diversion. EEG power spectrum changes and neural fuzzy system have been used to determine the drowsiness degree effectually, but it is not suitable for a wearable hardware implementation. EEG Alpha waves detection methods have been proposed via tracking two alpha-related phenomena to represent sleepiness levels, and marketing them using a support vector machine (SVM), however, the implementation result in very high power consumption critical to the on-body chip limits.

### DESCRIPTION OF THE INVENTION

The disclosed system is a simplified adaptive AIS preloaded with sodium chloride and lactic acid, Glossokinetic (GKP), electrocardiogram (EKG), electrooculography (EOG), electromyography (EMG) artifacts signature, and monitoring of the Alpha rhythm dynamics In addition, the disclosed system provides online-unsupervised learning of the drowsiness onset prefixing signals, without any offline training period required, because the attenuation of the Alpha rhythm and its duration within the epoch are the definite markers.

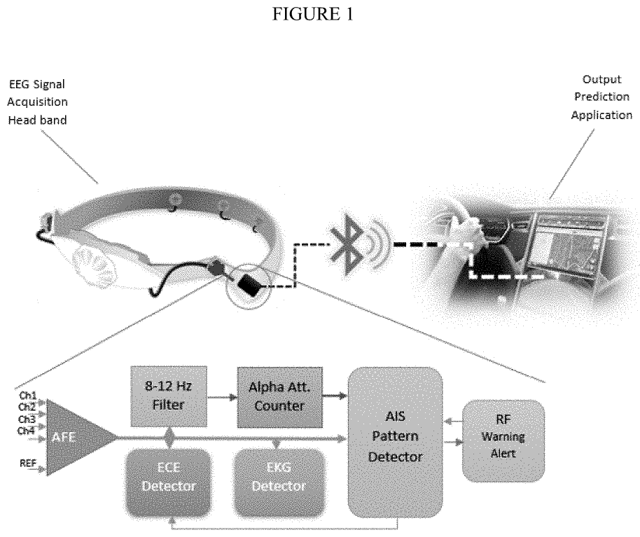

The system comprises three components: the dry electrodes (O1, O2, AF3 and AF4) on the headset, the detector chip, and the RF connection to the autonomous car system, as shown in FIG. 1.

The system is implemented in Verilog and has been tested in ModelSim, using 64 hours of life data from five adult subjects in a driving car environment that is verified to the sleep marks that accurately and provide early detection of sleep 164.45% faster than previous devices. This device is a step closer towards safer drowsy free roads.

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies. Although the terms “step” and/or “block” or “module” etc. might be used herein to connote different components of methods or systems employed, the terms should not be interpreted as implying any particular order among or between various steps herein disclosed unless and except when the order of individual steps is explicitly described.

Furthermore, the described features, structures, or characteristics may be combined in any suitable manner in one or more embodiments. Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment.

Moreover, the terms “substantially” or “approximately” as used herein may be applied to modify any quantitative representation that could permissibly vary without resulting in a change to the basic function to which it is related.

We claim:

1. A system for detecting drowsiness in a human driver, said system comprising:
(a) a head set comprising at least one dry electrode;
(b) a detector chip in communication with said head set; and
(c) an RF connection that operably connects said detector chip to an autonomous car system.

2. The system of claim 1, wherein the detector chip monitors at least one Alpha rhythm dynamic of said human driver.

3. The system of claim 1, wherein the detector chip monitors signals from the headset, said signals providing information on the human driver, said information comprising:
(a) sodium chloride and lactic acid levels;
(b) Glossokinetic;
(c) electrocardiogram;
(d) electrooculography;
(e) electromyography; and
(f) Alpha rhythm dynamics.
